(19) United States
(12) Patent Application Publication
(10) Pub. No.: US 2006/0177913 A1
(43) Pub. Date: Aug. 10, 2006

(54) Process for enantioselective enzymatic reduction of keto compounds

(75) Inventors: Christian Peschko, Munchen (DE); Jurgen Stohrer, Pullach (DE)

Correspondence Address: BROOKS KUSHMAN P.C., 1000 TOWN CENTER, TWENTY-SECOND FLOOR, SOUTHFIELD, MI, 48075, US

(73) Assignee: Consortium fur Elektrochemische Industrie GmbH, Munich (DE)

(21) Appl. No.: 11/053,011

(22) Filed: Feb. 8, 2005

(51) Int. Cl. C12P 7/04

(52) U.S. Cl. 435/157

(57) ABSTRACT

Chiral secondary alcohols may be produced enzymatically in high space-time yields while minimizing enzyme use, by reducing a keto compound in an aqueous reaction medium containing water, reducing agent, alcohol dehydrogenase and coenzyme, extracting the secondary alcohol formed by means of a further phase containing a water-immiscible organic solvent, and removing the phase used for extraction and reusing the aqueous reaction medium in step a).

### BACKGROUND OF THE INVENTION

1. Field of the Invention

The present invention relates to an enzymatic process for enantioselective reduction of organic keto compounds to give the corresponding chiral hydroxy compounds.

2. Background Art

Optically active hydroxy compounds are valuable synthetic building blocks for the preparation of important compounds with pharmacological action and other valuable properties. These compounds are often difficult to prepare by traditional chemical processes and the required optical purities for applications in the pharmaceutical or agrochemical sector can be achieved in this way only with difficulty. Therefore, biotechnological processes are increasingly employed in preparing chiral compounds, the stereoselective reaction being carried out by whole microorganisms or using completely or partially purified isolated enzymes.

Dehydrogenases and in particular alcohol dehydrogenases (ADH) are valuable catalysts for obtaining chiral products by stereoselective reduction of organic keto compounds to the corresponding chiral alcohols. Known enzymes are essentially the corresponding enzymes from yeast, equine liver or Thermoanaerobium brockii. These enzymes require NADH (nicotinamide adenine dinucleotide) or NADPH (nicotinamide adenine dinucleotide phosphate) as a coenzyme. Other examples of known alcohol dehydrogenases are an(S)-specific alcohol dehydrogenase from Rhodococcus erythropolis and an (R)-specific alcohol dehydrogenase from the genus Lactobacillus. Both enzyme species act on a broad spectrum of keto compound substrates and have high enantioselectivity. The alcohol dehydrogenases from Lactobacillus kefir (DE 40 14 573 C1) and Lactobacillus brevis (DE 196 10 984 A1) are particularly suitable for obtaining chiral (R)-alcohols.

Processes for stereoselective reduction of organic keto compounds by alcohol dehydrogenases to give the corresponding chiral alcohols, in which processes the keto compound is contacted with water, enzyme, coenzyme and a reducing agent required for regenerating the enzyme system, are known from the prior art. A disadvantage of these enzyme-catalyzed processes is the high cost of enzymes which is caused by highly specific enzyme use. Therefore, work is usually carried out in the prior art by using very small amounts of enzyme. The space-time yields achieved by this kind of process are universally low and, due to high process costs, are realized neither economically nor industrially.

Thus, WO 02/064579 A1 describes a process in which, for example, an amount of enzyme of 0.5 units/ml (U/ml) is used for the ADH-LB-catalyzed reduction of alkynones, thereby achieving space-time throughputs of only about 15 mol/m³ d, with about 60 kU of enzyme being consumed per mole of product. The unit mol/m³ d is the amount of product in moles obtained in a production process per cubic meter of reaction volume per day.

A process described in DE 196 10 984 A1 for the reduction of ketones with ADH-LB achieves a space-time throughput of 4.75 mol/m³ d with 4.5 U/ml enzyme used and about 950 kU of enzyme per mole of product being consumed.

Due to the often insufficient solubility of the organic substrates in an aqueous environment, use is also made of two-phase systems which, in addition to water, also contain an organic phase. However, the presence of an organic phase has, to a varying degree, a destabilizing effect on enzyme activity (M. V. Filho, T. Stillger, M. Müller, A. Liese, C. Wandrey, ANGEW. CHEM., 115, 3101-3104 (2003)).

Processes involving two-phase systems have partly improved space-time throughputs. However, enzyme consumption usually is also high. Therefore, processes of this kind can also be used economically and industrially only with limitation.

WO 02/086126 A2 discloses a process for ADH-LB-catalyzed reduction of a β-keto ester in a two-phase system, which consumes between 22.5 and 27.4 kU of enzyme per mole of product with space-time throughputs of from 200 to 350 mol/m³ d.

### SUMMARY OF THE INVENTION

It is therefore the object of the invention to provide a process for enzyme-catalyzed preparation of chiral alcohols with high space-time throughput and, at the same time, low enzyme consumption. These and other objects are achieved by a process for enzyme-catalyzed reduction of keto compounds, using a reaction medium comprising alcohol dehydrogenase, water, coenzyme, and reducing agent, from which, after reduction of the keto compound in a first step, the reaction products are extracted in a second step with an organic solvent. After extraction, the organic phase containing the reaction products is removed and the aqueous reaction medium is used again and the process is repeated.

### DETAILED DESCRIPTION OF THE PREFERRED EMBODIMENT(S)

The invention thus relates to a process for preparing chiral secondary alcohols, comprising the steps of
a) reducing a keto compound in an aqueous reaction medium containing water, reducing agent, alcohol dehydrogenase and coenzyme,
b) extracting the secondary alcohol thusly formed by means of a further phase containing a water-immiscible organic solvent,
c) removing the phase used for extraction and reusing the aqueous reaction medium in step a).

The process of the invention is characterized by high space-time throughputs with, at the same time, low enzyme consumption, enantiomeric purities of up to 99.9% with respect to the chiral hydroxy compounds prepared, and chemical yields of up to 99% or higher, based on the amount of keto compound used. The continuous presence of a potentially enzyme-deactivating organic solvent phase, as is the case in the known two-phase processes, is avoided. The process of the invention furthermore can be implemented with very simple apparatus, and thus industrially.

The actual reduction according to step a) is carried out in an aqueous system, without the simultaneous presence and/or addition of another phase which is used merely for extraction and which contains a water-immiscible organic solvent. Accordingly, the process of the invention dispenses with the specific addition of an organic extraction phase continually present in the system.

However, the formation of a separate phase in addition to the aqueous phase (reaction medium) at the onset, or during the reduction, cannot be ruled out. Such a separate phase, for example, may result from immiscibility of the substrate to be reduced (keto compound) with the aqueous reaction medium, which may be present only initially, or may persist over time.

Reactants which may be used are in general ketones, preferably those having from 3 to 40 carbon atoms.

In a preferred embodiment of the process of the invention, prochiral ketones of the general formula (I)
R¹—C(O)—R² (I)
are used, in which
R¹ and R² are selected independently of one another from the group of C₁-C₂₀-alkyl, C₃-C₂₀-cycloalkyl, C₅-C₂₀-aryl, C₁-C₂₀-heteroaryl, C₂-C₂₀-alkenyl, C₅-C₂₀-aralkyl, C₅-C₂₀-alkylaryl or R¹ and R² together can form a ring, and optionally, independently of one another, may be substituted with one or more radicals Z, where
Z is selected from the group of fluoro, chloro, bromo, iodo, —CN, —NO₂, —NO, —NR³OR³, —CHO, —SO₃, —COOH or —R³ and
R³is hydrogen or may have the meaning of R¹, and
R¹ and R², independently of one another, optionally have one or more methylene groups replaced by identical or different groups Y, where
Y is selected from the group of —CR³═CR³—,
—C≡—C—, —C(O)O—, —C(O)O—, —OC(O)O—, —C(O)OC(O)O—, —O—,
O—O—, —CR³═—, —C(O)—NR³—, —N═—, —NR³—NR³—, —NR³—O—,
—NR³—, —P(O)(OR³)O—, —OP(O)(R³)O—, —P(R³)—, —P(O)(R³)—,
—S—, —S—S—, —S(O)O—, —S(O)₂—, —S(O)NR³—, —S(O)(OR³)O—,
—Si(R³)₂—, —Si(R³)₂O—, —Si(R³)(OR³)—, —OSi(R³)₂O—, —OSi(R³)₂—
or —Si(R³)₂OSi(R³)₂—.

Preferred C₅-C₂₀-aryl or C₁-C₂₀-heteroaryl radicals for R¹ and R² are selected in particular from the group comprising phenyl, naphthyl, indolyl, benzofuranyl, thiophenyl, pyrrolyl, pyridinyl, imidazolyl, oxazolyl, isoxazolyl, furanyl and thiazolyl.

The compounds of the general formula (I) can in general also be used in the form of their salts.

Particularly preferred compounds of the general formula (I) are selected from the class of β-keto esters, α-keto esters, γ-keto esters, β-diketones, γ-diketones, σ-diketones, β-haloketones, α-haloketones, α,α-dihaloketones, α-alkoxy ketones, α-acyloxy ketones, β-alkoxy ketones, α-alkynyl ketones, alkenyl ketones, α-diketones, α,α-dialkoxy ketones, aryl ketones, and heteroaryl ketones.

Particularly suitable compounds of the general formula (I) are methyl 3-oxobutanoate, ethyl 3-oxobutanoate, methyl 4-chloro-3-oxobutanoate, ethyl 4-chloro-3-oxobutanoate, methyl 3-oxopentanoate, ethyl 3-oxopentanoate, 1-chloropropan-2-one, 1,1-dichloropropan-2-one, 3-oxobutan-2-one, 2,6-dimethylhexane-3,5-dione, 2,7-dimethylhexane-3,6-dione, 2,4-hexanedione, 2,4-pentanedione, tert-butyl 3-oxobutanoate, 2,5-hexanedione, 4-trimethylsilyl-3-butyn-2-one, 4-triisopropylsilyl-3-butyn-2-one, 1-chloro-4-trimethylsilyl-3-butyn-2-one, 1-chloro-4-triisopropylsilyl-3-butyn-2-one or 1-chloro-butyn-2-one, 1-chlorobutan-3-one, butanone, pentan-2-one, hexan-2-one, heptan-2-one, octan-2-one, 3-penten-2-one, 1-acetoxypropan-2-one, cyclopent-2-en-1-one, methyl 3-oxotetradecanoate, methyl 3-oxododecanoate, ethyl 2-oxopropionate, 1,4-dichlorobutanone, acetophenone, 3-methylbutanone, 1-benzyloxypropan-2-one and 2-methylcyclopentanone.

The compounds of the general formula (I) are used in the process of the invention in an amount of from 1% to 50%, based on the total volume of each reaction mixture, preferably from 3% to 25%, and in particular from 5% to 15%.

The reaction mixture should generally have a pH of from 5 to 10, preferably from 6 to 9.

The aqueous phase (reaction medium) preferably contains a buffer, in particular a potassium phosphate/potassium hydrogen phosphate, tris(hydroxymethyl)aminomethane/HCl, or triethanolamine/HCl, buffer having a pH from 5 to 10, preferably a pH from 6 to 9. The buffer concentration should preferably be from 5 mM to 150 mM.

The aqueous phase may additionally also contain magnesium ions, for example in the form of MgCl₂ added at a concentration of from 0.2 mM to 10 mM, preferably 0.5 mM to 2 mM, based on the amount of water used. Additionally, the aqueous phase may contain further salts, for example NaCl, and other additives, such as dimethyl sulfoxide, glycerol, glycol, ethylene glycol, sorbitol, mannitol, or sugar.

Coenzymes which may be used are, for example, NADP, NADPH, NAD, NADH or salts thereof. The concentration of coenzyme in the aqueous phase is preferably from 0.01 mM to 0.25 mM, more preferably from 0.02 mM to 0.1 mM.

The reducing agent added to the aqueous phase is, in general, a secondary alcohol, preferably isopropanol. The amount of alcohol added to each mixture is from 1% to 60%, based on the total volume of the mixture, preferably 1% to 50%, more preferably 1% to 30%, and most preferably 4% to 15%. The reducing agent may also be formic acid or the salts of formic acid, in particular sodium formate, and this may require, where appropriate, the addition of additional substances such as formate dehydrogenase (FDH), for example.

Suitable alcohol dehydrogenases are derived, for example, from yeast, equine liver or Rhodococcus erythropolis, with these enzymes requiring the coenzyme NADH, or from Thermoanaerobium spec., Lactobacillus kefir or Lactobacillus brevis, with these enzymes requiring the coenzyme NADPH. However, other alcohol dehydrogenases may be used as well. The alcohol dehydrogenase may be either completely or partially purified or may be present in cells. The cells used in this process may be native or may have been permeabilized or lysed.

The volume activity of the alcohol dehydrogenase used is from 100 units/ml (U/ml) to 5000 U/ml, preferably about 1000 U/ml. 50,000 to 700,000 U of alcohol dehydrogenase (ADH) are available for converting in each case 1 kg of compound of the general formula (I) in the aqueous phase, and is reused all or in part after extraction of the product. Particular preference is given to using more than 15 U/ml ADH in the aqueous phase.

The temperature of the reaction mixture is preferably from 0° C. to 60° C., particularly preferably from 20° C. to 40° C., and at a pressure of from 10 mbar to 5 bar, more preferably at a pressure of from 30 mbar to 1 bar. The reaction time is between 30 min and 50 h, preferably 1 h to 20 h, depending on the type and amount of alcohol dehydrogenase used and compound of the general formula (I) used. During the course of the reaction, further reducing agents or reactants may be added, in particular reactants in the form of ketones of the general formula (I).

Extraction with the water-immiscible extraction phase containing an organic solvent may be carried out batchwise or continuously. In the many possible variants of the extraction step, extraction may involve the entire batch, or in particular with continuous extraction, only a portion of the aqueous reaction medium. The batchwise or, preferably, continuous extraction with a second phase containing a water-immiscible organic solvent is conducted in a conventional manner, known to those skilled in the art. Incomplete extraction of the products from the aqueous reaction mixture is also sufficient for the process of the invention.

According to the inventive process, the further phase which has been obtained by addition of a water-immiscible organic solvent, in the case of batchwise extraction, is present only in the second step, after reduction has been carried out, for the purpose of extracting the desired reaction products, in contrast to the 2-phase processes known from the prior art.

In the case of continuous extraction, the aqueous reaction medium of one or more reaction vessels is, according to the process of the invention, contacted with the organic extraction phase in a separate, connected extraction apparatus. Preferably, only part of the reaction medium is extracted. In a particularly preferred embodiment, the reaction medium is contacted with the extraction phase and separated again (phase separation) in portions and with constant flow in a separate part of the apparatus. In one embodiment of constant flow continuous extraction, an extraction column is used in which the aqueous phase and the extraction phase are contacted in a countercurrent process in a vertical column, preferably a glass column, which contains sieve trays resulting in eddying and/or mixing of the phases. Continuous extraction is particularly preferable, if the process of the invention is intended to be operated continuously over a long period of time.

In a particularly preferred embodiment, reduction in the aqueous phase according to step a) is carried out in a plurality of reaction vessels in parallel and the aqueous reaction medium is then completely or partially removed from one or more vessels and extracted separately in the manner described above. The extracted aqueous phase may then be reacted further immediately, while portions which have not been extracted yet are continuously fed to the extraction and subsequently to a different vessel. In this way it is possible to process a plurality of reaction mixtures sequentially and in parallel in a plurality of reaction vessels. At the same time, unused standing times (“idle times”) of the aqueous enzyme-containing reaction medium are also avoided in this manner. Consequently, only that part of the aqueous reaction medium, which is undergoing step b) (extraction), is momentarily not productive.

It is preferable to establish a contact time between the aqueous phase (reaction medium) and the organic extraction phase, which is as short as possible, independently of whether the extraction is carried out batchwise or continuously. Particular preference is given to contact times between 1 min and 60 min, especially from 1 min to 10 min.

Suitable organic solvents are any water-immiscible solvents which are capable of isolating the secondary alcohol formed from the aqueous phase, preferably organic solvents selected from the group of esters and/or ethers, and/or alkanes. Particular preference is given to using ethyl acetate, methyl acetate, propyl acetate, isopropyl acetate, butyl acetate, tert-butyl acetate, diethyl ether, diisopropyl ether, dibutyl ether and tert-butyl methyl ether (MTBE), n-pentane, n-hexane, n-heptane, or mixtures thereof. Very particular preference is given to using MTBE.

After the organic extraction phase has been removed, it is preferably worked up by means of distillation, in which process the reaction product is concentrated and byproducts are removed from the extraction solvent partially or to completion, it being possible for the solvent to be reused for extraction, e.g., for repeated or continuous extractions.

The aqueous phase remaining after extraction is again admixed with reactant of the general formula (I) (keto compound) and reducing agent and incubated. In this context, it is possible to add, if required, additional enzyme and coenzyme prior to incubation.

In a particularly preferred embodiment, reduction is carried out at reduced pressure, usually at a pressure of from 1 mbar to about 1 bar, preferably 1 mbar to 100 mbar, and more preferably from 30 mbar to 70 mbar, and the volatile components in this process are continuously removed from the reaction system. This procedure is particularly suitable when the reducing agent used is isopropanol and volatile components such as acetone are removed continuously from the reaction mixture. Surprisingly, a particularly good extraction performance and reusability of the extraction phase are achieved in this manner.

The desired final product is obtained by purifying the organic extraction solution containing the crude product, for example by means of fine distillation. The products obtained in this way are typically characterized by yields of >95% and purities, as enantiomeric excess (ee) of >99%.

The process of the invention enables an aqueous, enzyme-containing reaction medium for reducing ketones to chiral alcohols, to be reused. The resulting high space-time throughputs and the low amount of enzyme consumed per mole of product, due to repeated use of the enzyme solution, make possible a cost-effective enzymatic reduction of ketones to chiral alcohols. Moreover, reuse makes it possible to dispense with a complicated complete extraction of the product and instead to carry out only a partial, less complicated extraction. This also makes it possible to drastically reduce the aqueous phase requiring disposal. The particularly preferred embodiments moreover ensure a particularly efficient extraction of product with low consumption of extraction solvent.

The processes known from the prior art for enzyme-catalyzed preparation of chiral alcohols starting from prochiral ketones, independently of whether they are one-phase or two-phase processes, give no indication whatsoever to the skilled worker of the fact that the process of separating reduction and extraction, in combination with recycling the aqueous phase, results in significant advantages of the kind mentioned.

The following examples illustrate the invention:

### EXAMPLE 1a

### Reaction of Acetoacetic Ester in the Aqueous Phase

Procedure

400 ml of a solution of water, phosphate buffer (50 mM), isopropanol (1.0 M), 25 U/ml ADH-LB (crude extract), NADP disodium salt (0.0415 mM) and acetoacetic ester (0.5 M), of pH 6.5 was introduced to a 500 ml round-bottomed flask with a magnetic stirrer and reflux condenser and stirred vigorously at 30° C. After 19 hours, the solution was extracted with 4×400 ml of methyl tert-butyl ether (MTBE) and the product was isolated by evaporating the organic phases. The yield by weight of ethyl (R)-3-hydroxybutanoate was determined by means of GC and NMR spectroscopy. A product yield of 190 mmol (95%) and a space-time throughput of 25 mol/m³ h were realized.

### EXAMPLE 1b

### Reuse of the Extracted Aqueous Phase

Procedure

The aqueous phase of example 1a which remained after extraction with MTBE, was admixed with isopropanol (400 mmol) and acetoacetic ester (200 mmol) and adjusted to pH 6.5 and then introduced to a 500 ml round-bottomed flask with magnetic stirrer and reflux condenser and stirred vigorously at 30° C.

After 19 hours, the solution was extracted with 4×400 ml of MTBE and the product was isolated by evaporating the organic phases. The yield by weight of ethyl (R)-3-hydroxybutanoate was determined by means of GC and NMR spectroscopy. A product yield of 190 mmol (95%) and a space-time throughput of 25 mol/m³ h were realized.

### COMPARATIVE EXAMPLE 2a

### Reaction of Acetoacetic Ester in a Two-Phase Mixture

Procedure

200 ml of a solution of water, phosphate buffer (50 mM), isopropanol (1.0 M), 25 U/ml ADH-LB (crude extract), NADP disodium salt (0.0415 mM) and acetoacetic ester (0.5 M), of pH 6.5 was admixed with 200 ml of MTBE and introduced to a 500 ml round-bottomed flask with a magnetic stirrer and reflux condenser and stirred vigorously at 30° C.

After 19 hours, the organic phase was removed, the aqueous solution was extracted with 3×200 ml of MTBE and the product was isolated by evaporating the organic phases. The yield by weight of ethyl (R)-3-hydroxybutanoate was determined by means of GC and NMR spectroscopy. A product yield of 83 mmol (83%) and a space-time throughput of 10.9 mol/m³ h were realized.

### COMPARATIVE EXAMPLE 2b:

### Reuse of the Extracted Aqueous Phase

Procedure

The aqueous phase of example 2a, which remained after extraction with MTBE, was admixed with isopropanol (200 mmol) and acetoacetic ester (100 mmol), adjusted to pH 6.5, admixed with 200 ml of MTBE, and introduced to a 500 ml round-bottomed flask with magnetic stirrer and reflux condenser and stirred vigorously at 30° C.

After 19 hours, the organic phase was removed, the aqueous solution was extracted with 3×200 ml of MTBE, and the product was isolated by evaporating the organic phases. The yield by weight of ethyl (R)-3-hydroxybutanoate was determined by means of GC and NMR spectroscopy. A product yield of 85 mmol (85%) and a space-time throughput of 11.2 mol/m³ h were realized.

TABLE 1

| | Example 1a | | Comparative | Comparative |
|---|---|---|---|---|
| | 1-phase/ | Example 1b | Example 2a | Example 2b |
| | first | 1-phase/ | 2-phase/ | 2-phase/ |
| | mixture | reuse | first mixture | reuse |
| Yield | 95% | 95% | 83% | 85% |
| Space-time | 25 mol/m³h | 25 mol/m³h | 10.9 mol/m³h | 11.2mol/m³h |
| throughput | | | | |

The one-phase system, both in the first mixture and in the reused extracted aqueous phase, shows better substrate conversion and a distinctly higher space-time throughput than the two-phase system obtained by addition of MTBE.

### EXAMPLE 3

### Enzyme-Catalyzed Preparation of Ethyl (R)-3-Hydroxy-Butanoate on the 100 l Scale With Reuse of the Enzyme-Containing Aqueous Phase

The enzyme-catalyzed synthesis of ethyl (R)-3-hydroxy-butanoate involved the use of recombinant alcohol dehydrogenase from Lactobacillus brevis (=ADH-LB) as a crude extract with an average volume activity of 1.08 kU/ml and of β-NADP disodium salt (97% chemical purity) as coenzyme.

3A. First Mixture

TABLE 2

| Substance | Amount or Volume | |
|---|---|---|
| Water | 84.1 | |
| NaCl | 0.84 | kg |
| KOH* | approx. 0.39 | kg |
| Phosphoric acid (85% strength) | 0.29 | l |
| MgCl₂.6H₂O | 17.1 | g |
| Isopropanol | 7.65 | l |
| Ethyl acetoacetate | 6.51 | kg |
| NADP disodium salt | 3.27 | g |
| ADH-LB(1.08 kU/ml) | 2.3 | l |

*for adjusting to pH 6.5, therefore no exact amount can be given.

The total volume of the aqueous mixture was approx. 100 l.

In an enameled tank with stirrer, the solution of magnesium chloride, sodium chloride, phosphoric acid and water was adjusted to a pH of 6.5 by adding KOH, followed by adding the NADP salt and the enzyme crude extract. After heating the mixture to 30° C., ethyl acetoacetate and isopropanol were added.

The mixture was stirred for approx. 17 h, until conversion was >97%, according to GC or NMR analysis, with the temperature of the mixture being kept between 20 and 30° C. and the pH continuously monitored.

The aqueous phase was continuously extracted in a countercurrent process with 400 l of tert-butyl methyl ether (MTBE) (flow rate of extraction phase (organic phase)/flow rate of reaction medium (aqueous phase) approx. 4/1) and the extracted aqueous phase was recycled into the reaction tank for reuse. In this process, the organic phase was continuously redistilled so that isolated ethyl (R)-3-hydroxybutanoate remained entirely in the distillation residue. The phases separated instantly and virtually completely in the extraction process.

3B. First Reuse of the Aqueous Phase

The extracted aqueous solution was treated with 7.65 l of isopropanol and 6.51 kg of ethyl acetate. ADH-LB crude extract was added to the mixture. In this example, the added amount was always 10% of the initial amount, i.e. in each case about 25 kU of ADH-LB. Likewise, in each case 10% of the initial amount, i.e. 0.327 g of NADP disodium salt, were always added for reuse in this example. If necessary, the pH was adjusted to 6.5 by adding KOH. The reaction was carried out in a manner similar to example 3A, and the aqueous solution was extracted in a manner similar to example 3A with redistilled MTBE from example 3A.

3C. Second Reuse of the Aqueous Phase

Additions to the extracted aqueous solution of example 3B were carried out in a manner similar to example 3B, and the solution was reacted in a manner similar to example 3A.

Since experience shows that the rate of phase separation decreases after repeated use of redistilled MTBE, this extraction phase was discarded, after the product had been worked up by distillation and removed, and replaced by 400 l of fresh MTBE.

3D. Third to Fifth Reuse of the Aqueous Phase

The reactions were carried out in a manner similar to examples 3B and 3C, and the extractions were carried out in a manner similar to examples 3A to 3C. When the rate of phase separation decreased, the redistilled organic extraction phase was replaced with fresh MTBE, in each case in a similar manner.

TABLE 3
(Summary of the results of examples 3A to 3D):

| Example 3 | Time (h) | Conversion (%) | ee |
|---|---|---|---|
| First mixture (3A) | 17 | 99 | >99% |
| 1st reuse (3B) | 17 | 98 | >99% |
| 2nd reuse (3C) | 16 | 97 | >99% |
| 3rd reuse (3D/1) | 17 | 98 | >99% |
| 4th reuse (3D/2) | 17 | 98 | >99% |
| 5th reuse (3D/3) | 17 | 98 | >99% |

3E. Distillation of the Crude Product

After MTBE and isopropanol have been removed completely and substantially, respectively, fractional distillation produced 38 kg (96%) of ethyl (R)-3-hydroxybutanoate with >99% chemical purity and an optical purity of >99% ee.

TABLE 4
Example 3

| | | | |
|---|---|---|---|
| Substrate used: | | 39.06 | kg (300 mol) |
| Yield: | 96% | 38.02 | kg (288 mol) |
| Space-time throughput: | 28.5 mol/m³h | 684 | mol/m³d |
| Enzyme consumption: | 1.25 MU | 4.3 | kU/mol of product |
| Coenzyme Consumption: | 6.23 mmol | 17 | mg/mol of product |

### EXAMPLE 4

### Enzyme-Catalyzed Preparation of Ethyl (R)-3-Hydroxy-Butanoate With Reuse of the Enzyme-Containing Aqueous Phase at Reduced Pressure

4A. First Use

The same amount or volume of the same reagents as in example 3A were used (see table 2), and the reaction mixture was prepared in a manner similar to example 3A.

After closing the reaction tank, a pressure of approx. 60 mbar was established above the reaction mixture in the interior by applying vacuum. The mixture was stirred for approx. 17 h, until conversion was >97%, according to GC or NMR analysis, with the temperature of the mixture being kept between 20 and 30° C. and the pH continuously monitored.

The aqueous solution was continuously extracted in a countercurrent process with 400 l of tert-butyl methyl ether (MTBE) (flow rate of extraction phase (organic phase)/flow rate of reaction medium (aqueous phase) approx. 4/1). In this process, the organic phase was continuously redistilled so that isolated ethyl (R)-3-hydroxybutanoate remained entirely in the distillation residue. The extracted aqueous phase was recycled into the reaction tank for reuse. The phases separated instantly and completely in the extraction process.

4B. Reuse of the Aqueous Phase

The extracted aqueous solution was treated with 7.65 l of isopropanol and 6.51 kg of ethyl acetate. ADH-LB crude extract was added to the mixture. In this example, the added amount was always 10% of the initial amount, i.e. in each case about 25 kU of ADH-LB. Likewise, in each case 10% of the initial amount, i.e. 0.327 g of NADP disodium salt, were always added for reuse in this example. If necessary, the pH was adjusted to 6.5 by adding KOH.

The reaction as well as the workup were carried out in a manner similar to example 4A. The reuse of the aqueous phase was carried out in a similar manner 5 times altogether (examples 4B/a-4B/e).

When the process of the invention is carried out under reduced pressure, the acetone content of MTBE is kept below the critical limit and the phases separated in the extraction processes in each case instantly and completely. The extraction phase may be reused in this manner as often as desired.

TABLE 5
(Summary of the results of examples 4A and 4B/a to 4B/e):

| Example 4 | Time (h) | Conversion (%) | ee |
|---|---|---|---|
| First mixture (4A) | 17 | 99 | >99% |
| 1st reuse (4B/a) | 17 | 98 | >99% |
| 2nd reuse (4B/b) | 16 | 97 | >99% |
| 3rd reuse (4B/c) | 17 | 98 | >99% |
| 4th reuse (4B/d) | 17 | 98 | >99% |
| 5th reuse (4B/e) | 17 | 98 | >99% |

4C. Distillation of the Crude Product

After MTBE and isopropanol have been removed completely and substantially, respectively, fractional distillation produced 38.4 kg (97%) of ethyl (R)-3-hydroxybutanoate with >99% chemical purity and an optical purity of >99% ee.

TABLE 6
Example 3

| | | | |
|---|---|---|---|
| Substrate used: | | 39.06 | kg (300 mol) |
| Yield: | 97% | 38.42 | kg (291 mol) |
| Space-time throughput: | 28.8 mol/m³h | 691 | mol/m³d |
| Enzyme consumption: | 1.25 MU | 4.3 | kU/mol of product |
| Coenzyme Consumption: | 6.23 mmol | 17 | mg/mol of product |

The reuse of the extracted aqueous phase delivered consistently high conversions and enantiomeric excesses.

It was possible to reuse the redistilled extraction solvent without limitation.

TABLE 7

| | Example 3 | Example 4 |
|---|---|---|
| Conversion of Aqueous | no | yes |
| Phase at Reduced | | |
| Pressure | | |
| Reusability of the | 2× | unlimited |
| extraction solvent | | |

### EXAMPLE 5

### Enzyme-Catalyzed Preparation of (S)-2-Hexanol on the 4000 l Scale With Reuse of the Enzyme-Containing Aqueous Phase

The enzyme-catalyzed synthesis of (S)-2-hexanol involved the use of recombinant alcohol dehydrogenase from Thermoanaerobium spec. (=ADH-T) as crude extract with an average volume activity of 545 U/ml and of β-NADP disodium salt (97% chemical purity) as coenzyme.

5A. First Mixture

TABLE 8

| Substance | Amount or Volume | |
|---|---|---|
| Water | 2960 | l |
| NaOH* | approx. 9.8 | kg* |
| Phosphoric acid (85% strength) | 17.6 | kg |
| MgCl2 × 6H20 | 600 | g |
| Isopropanol | 800 | l |
| 2-Hexanone | 199.5 | kg |
| NADP disodium salt | 160 | g |
| ADH-T (545 U/ml) | 22 | l |

*for adjusting to pH 7.0, therefore no exact amount can be given.

The total volume of the aqueous mixture was approx. 4000 l.

In an 8000 l enameled tank with stirrer, the solution of magnesium chloride, phosphoric acid and water was adjusted to a pH of 6.5 by adding NaOH, followed by the addition of 2-hexanone, isopropanol, NADP salt and enzyme crude extract. The pH was adjusted to 7.0 by adding NaOH and the mixture was then heated to 30° C.

The mixture was stirred until conversion was 74%, according to GC analysis.

The aqueous phase was extracted twice with n-heptane (600 l and 400 l) and the extracted aqueous phase was recycled into the reaction tank for reuse and stirred in vacuo (<100 mbar) with a nitrogen sparge for several hours. The organic extract was intermediately stored.

5B. Reuse of the Aqueous Phase

The extracted aqueous solution was treated with 573 l of isopropanol and 160 kg of 2-hexanone. The pH was set to pH 7.0 with NaOH and the temperature was adjusted to 30° C. The reaction was carried out in a manner similar to example 5A. The mixture was stirred until conversion was 70%, according to GC analysis. The subsequent extraction was carried out in a manner similar to example 5A.

5C. Purification of the Heptane Extract

The pooled heptane extracts were concentrated, after water had been azeotropically removed, and then fractionally distilled. Here, a total of 216 kg (59% of theory) of (S)-2-hexanol of >99% ee were obtained.

TABLES 9 + 10
(Summary of the results of examples 5A to 5C):

| Reaction | Conversion (%) | ee |
|---|---|---|
| First mixture (5A) | 74 | >99% |
| Reuse (5B) | 70 | >99% |

| Distillation (5C) | Yield | % of theory | ee |
|---|---|---|---|
| (S)-2-Hexanol | 216 kg | 59 | >99% |

### EXAMPLE 6

### Enzyme-Catalyzed Preparation of (S)-2-Pentanol on the 3000 l Scale

The enzyme-catalyzed synthesis of (S)-2-pentanol involved the use of recombinant alcohol dehydrogenase from Thermoanaerobium spec. (=ADH-T) as crude extract with an average volume activity of 545 U/ml and of β-NADP disodium salt (97% chemical purity) as coenzyme.

6A. First Mixture

TABLE 11

| Substance | Amount or Volume | |
|---|---|---|
| Water | 1200 | l |
| NaOH (aq., 25% w/v)* | approx. 10 | l* |
| Phosphoric acid (85% strength) | 5 | kg |
| MgCl₂ × 6H₂O | 183 | g |
| Isopropanol | 1350 | l |
| 2-Pentanone | 175 | kg |
| NADP disodium salt | 106 | g |
| ADH-T (5454 U/ml) | 14.7 | l |

*for adjusting to pH 7.0, therefore no exact amount can be given.

The total volume of the aqueous mixture was approx. 2700 l.

In a 5000 l enameled tank with stirrer, a solution of magnesium chloride, phosphoric acid, water and NaOH of pH 6.5 was admixed with 2-pentanone, isopropanol, NADP salt and the enzyme crude extract solution. The mixture was heated to 40° C. at pH 7.0.

The mixture was stirred until conversion was 66%, according to GC analysis.

The aqueous phase was extracted twice with n-pentane (1000 l and 300 l) and the extracted aqueous phase was recycled into the reaction tank for reuse and stirred in vacuo (<100 mbar) with a nitrogen sparge for several hours. The organic extract was intermediately stored.

6B. Reuse of the Aqueous Phase

The extracted aqueous solution was treated with 1350 l of isopropanol and 175 kg of 2-pentanone. The pH was adjusted to 7.0 with NaOH and the temperature was adjusted to 40° C. The reaction was carried out in a manner similar to example 6A. The mixture was stirred until conversion was 64%, according to GC analysis. The subsequent extraction was carried out in a manner similar to example 6A.

6C. Purification of the Pentane Extract

The pooled pentane extracts were concentrated, after water had been azeotropically removed, and then fractionally distilled. A total of 208 kg (58% of theory) of (S)-2-pentanol of >99% ee were obtained.

TABLES 12 + 13
(Summary of the results of example 6):

| Reaction | Conversion (%) | ee |
|---|---|---|
| First mixture (6A) | 66 | >99% |
| Reuse (6B) | 64 | >99% |

| Distillation (6C) | Yield | % of theory | ee |
|---|---|---|---|
| (S)-2-Hexanol | 208 kg | 58 | >99% |

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A process for preparing chiral secondary alcohols, comprising the steps of
a) enzymatically reducing a keto compound in an aqueous reaction medium containing water, reducing agent, alcohol dehydrogenase and coenzyme,
b) extracting the secondary alcohol formed by means of a further phase containing a water-immiscible organic solvent,
c) removing the phase used for extraction and reusing the aqueous reaction medium in step a).

2. The process of claim 1, wherein the reduction is carried out under reduced pressure and volatile components are removed from the reaction system.

3. The process of claim 1, wherein the reducing agent comprises isopropanol.

4. The process of claim 2, wherein the reducing agent comprises isopropanol.

5. The process of claim 1, wherein the reducing agent comprises formic acid or a salt of formic acid.

6. The process of claim 2, wherein the reducing agent comprises formic acid or a salt of formic acid.

7. The process of claim 1, wherein the phase used for extraction comprises methyl tert-butyl ether.

8. The process of claim 2, wherein the phase used for extraction comprises methyl tert-butyl ether.

9. The process of claim 3, wherein the phase used for extraction comprises methyl tert-butyl ether.

10. The process of claim 1, wherein the time of contact between the aqueous reaction medium and the organic extraction phase during extraction is from 1 min to 10 min.

11. The process of claim 2, wherein the time of contact between the aqueous reaction medium and the organic extraction phase during extraction is from 1 min to 10 min.

12. The process of claim 3, wherein the time of contact between the aqueous reaction medium and the organic extraction phase during extraction is from 1 min to 10 min.

13. The process of claim 7, wherein the time of contact between the aqueous reaction medium and the organic extraction phase during extraction is from 1 min to 10 min.

14. The process of claim 1, wherein the alcohol dehydrogenase comprises an alcohol dehydrogenase from yeast, equine liver, Rhodococcus erythropolis, Thermoanaerobium spec., Lactobacillus kefir or Lactobacillus brevis.

15. The process of claim 1, wherein the coenzyme comprises at least one of NADP, NADPH, NAD, NADH or salts thereof.

16. The process of claim 1, wherein the aqueous reaction mixture is contained in a first reaction vessel, and at least a portion of the aqueous reaction mixture is removed from the first reaction vessel, extracted with a further phase containing a water immiscible solvent, the aqueous phase and the further phase are phase separated, and the aqueous phase is returned to the first reaction vessel or to a further reaction vessel whereupon step a) is repeated.

17. The process of claim 2, wherein the aqueous reaction mixture is contained in a first reaction vessel, and at least a portion of the aqueous reaction mixture is removed from the first reaction vessel, extracted with a further phase containing a water immiscible solvent, the aqueous phase and the further phase are phase separated, and the aqueous phase is returned to the first reaction vessel or to a further reaction vessel whereupon step a) is repeated.

18. The process of claim 16, wherein the extraction is a batch extraction.

19. The process of claim 16, wherein the extraction is a continuous, countercurrent extraction.

20. The process of claim 1, wherein the keto compounds used are prochiral ketones of the general formula (I)
R¹—C(O)—R² (I),
in which
R¹ and R² are selected independently of one another from the group consisting of C₁-C₂₀-alkyl, C₃-C₂₀-cycloalkyl, C₅-C₂₀-aryl, C₁-C₂₀-heteroaryl, C₂-C₂₀-alkenyl, C₅-C₂₀-aralkyl, C₅-C₂₀-alkylaryl, and rings formed from R¹ and R²,
where R¹ and R², independently of one another, are optionally substituted with one or more radicals Z, where
Z is selected from the group consisting of fluoro, chloro, bromo, iodo, —CN, —NO₂, —NO, —NR³OR³, —CHO, —SO₃H, —COOH, and —R³ and
R³ is R¹ or hydrogen, and
in R¹ and R², independently of one another, one or more methylene groups may be replaced by identical or different groups Y, where
Y is selected from the group consisting of —CR³═CR³—,
—C≡C—, —C(O)—, —C(O)O—, —OC(O)—, —C(O)OC(O)—, —O—,
—O—O—, —CR³═N—, —C(O)—NR³—, —N═N—, —NR³—NR³—, —NR³—O—,
—NR³—, —P(O)(OR³)O—, —OP(O)(R³)O—, —P(R³)—, —P(O)(R³)—,
—S—, —S—S—, —S(O)O—, —S(O)₂—, —S(O)NR³—, —S(O)(OR³)O—,
—Si(R³)₂—, —Si(R³)₂O—, —Si(R³)(OR³)—, —OSi(R³)₂O—,
—OSi(R³)₂—, and —Si(R³)₂OSi(R³)₂—.
